(19) United States
(12) Patent Application Publication
(10) Pub. No.: US 2023/0372682 A1
(43) Pub. Date: Nov. 23, 2023

(54) IMPLANT DETACHMENT MECHANISM

(71) Applicant: SAHAJANAND MEDICAL TECHNOLOGIES LIMITED, Gujarat (IN)

(72) Inventors: Abhijeet SINGHVI, Gujarat (IN); Chirag Maheshbhai SOLANKI, Gujarat (IN); Shivanshu PANDEY, Uttar Pradesh (IN); Akashkumar Ashokbhai MUNJANI, Gujarat (IN); lsha VASHISHT, Haryana (IN)

(21) Appl. No.: 17/816,560

(22) Filed: Aug. 1, 2022

Related U.S. Application Data
(63) Continuation of application No. PCT/IN2022/050550, filed on Jun. 16, 2022.

(30) Foreign Application Priority Data
May 18, 2022 (IN) 202221028620

(51) Int. Cl. A61M 27/00; A61M 25/10; A61M 25/09

(52) U.S. Cl. CPC A61M 27/002; A61M 25/10; A61M 25/09041; A61M 2025/1054; A61M 2025/09116

(57) ABSTRACT

An implant holder for holding and controlling release of an implant from a delivery catheter is provided. The implant holder is disposed in the distal section of the delivery catheter. The implant holder engages the implant in the collapsed state for restraining axial and radial displacement of the implant. The implant holder includes specific invariant features in terms of uniquely designed releasing part and pusher block that are adapted to uniquely accommodate and disengage the implant from the delivery catheter.

### CROSS REFERENCE(S) To RELATED APPLICATIONS

This application is a continuation of PCT Application No. PCT/IN2022/050550, filed Jun. 16, 2022, which claims priority to Indian Application No. IN202221028620, filed May 18, 2022, the entire contents of each being hereby incorporated by reference.

### TECHNICAL FIELD

The technology described herein relates to a percutaneous implant delivery system and methods to deliver an implant inside a human or animal body. Specifically, the technology is related to an implant detachment mechanism for assisting in deployment of a coronary or a peripheral implant.

### BACKGROUND

Proper functioning of different organs is essential for proper functioning of a human or animal body. For example, a healthy heart along with healthy arteries, veins, implants, nodes, walls, and remaining constituents, is essential for proper functioning of the other organs and the cardiovascular system itself. However, due to factors like age, disease, infections or genetic disorder, the working efficiency of the organs reduce significantly and sometimes, that may be a severe and potentially life-threatening condition. Conventionally, surgery was one main option to address the severely diseased organs e.g., replacing the diseased valve by a mechanical implant or bypassing or removing a blocked artery using a harvested artery etc.

However, in recent years, an alternative less invasive transcatheter approach has been developed that delivers an implant using a percutaneous catheter transvascularly through variety of access points in a cardiovascular network e.g., through femoral artery, transapically, transaortic, trans-axillary etc. These implants may be, but not limited to, a stent, a valve, a mesh, a balloon, a patch, a drug-containing matrix, a shunt, or a combination thereof.

During the transvascular procedure, a catheter delivery system, carrying an implant, plays a vital role as the operator's maneuvering actions at proximal end (handle) of a delivery system directly impacts the positioning, movement of the distal section (tip and capsule), and performance of the implant after the deployment. The effect of maneuvering actions transfers through a catheter shaft from the proximal end to the distal end. The catheter shaft is situated between the proximal end and the distal end. However, sometimes, the implant doesn't get detached from the delivery system quickly and requires additional maneuvering that consumes additional time and may also reduce accuracy in positioning of the implant.

Hence, there is a need to provide a detachment mechanism in a catheter delivery system to trans-vascularly deliver an implant to avoid the shortcomings known in the art and specifically to provide a catheter delivery system that gives precision and efficiency in detachment and deployment of the implant.

### SUMMARY

The subject technology is illustrated, for example, according to various aspects described below.

According to certain example embodiments, an implant detaching mechanism to detach an implant from an implant holder, comprises a catheter having a guidewire shaft, an inner shaft, a handle and a rotary knob. A release block and a pusher block, combinedly forms the implant holder. The release block has a detaching end that has an impact surface, the pusher block has at least a pin, the pusher block is fixed on the inner shaft and the release block is fixed on the guidewire shaft. The relative movement between the inner shaft and the guidewire shaft causes the release block and the pusher block to move closer and apart. The pin is used for engaging the implant. On moving the pusher block and the release block towards each other, at one point, the impact surface at the detaching end of the release block impacts the pin and the resultant impact force contributes in detachment of the engaged implant from the pin.

According to certain example embodiments, an implant detaching mechanism to detach an implant from an implant holder, comprises a catheter having a guidewire shaft, an inner shaft, a rotary knob and a handle. A release block containing at least one leg and a pusher block containing at least one sliding slot, combinedly forms the implant holder, wherein the leg and the sliding slot are arranged in a sliding manner. Further, the release block comprises a releasing part that along its length has a symmetric or asymmetric shape. On moving the release block towards the pin and due to symmetric or asymmetric shape of the releasing part, a vertical force is applied on the engaging part of the implant frame and the engaging part comes out of the pin as the release block approaches the pin. In an alternative arrangement, the pusher block is movable due to longitudinally movable inner shaft and on moving the pusher block, the pin moves towards the release block and due to linear or non-linear shape of the releasing part, a vertical force is applied on the engaging part of the implant frame and the engaging part comes out of the pin as the release block approaches the pin.

According to certain example embodiments, an implant detaching mechanism to detach an implant from an implant holder, comprises a catheter having a guidewire shaft, an inner shaft, a rotary knob and a handle. A riser, a receiver and a movable pin, combinedly forms the implant holder. The riser and the receiver have at least one inclined end. The riser and the receiver are fixed on the guidewire shaft and their inclined ends face each other. The movable pin is made of cylindrical part and a hook, wherein the cylindrical part is slidingly movable in a slot in the inner shaft. The hook has two inclined surface, and the hook is situated, without any fixed connection, between the riser and the receiver, each inclined surface of the hook accommodates with the inclined surfaces of the riser and the receiver. Depending on the direction of the longitudinal movement of the guidewire shaft, due to rotational movement of the rotary knob in the handle, the cylindrical part of the movable pin moves through the slot in the inner shaft in vertical direction and the inclined surfaces of the hook slides on the inclined surfaces of the riser and the receiver.

It will be appreciated that the above-described features are merely examples and other features, aspects, and advantages of the subject matter are further illustrated in the figures and described in the corresponding description below.

### BRIEF DESCRIPTION OF ACCOMPANYING DRAWINGS

The detailed description is described with reference to the accompanying figures.

FIG. 1, illustrates a side view of an example catheter delivery system, according to some example embodiments;

FIG. 1A illustrates an isometric view of a handle of the catheter delivery system of FIG. 1, and FIG. 1B shows a cross-sectional view of the handle of the catheter delivery system of FIG. 1, according to certain example embodiments;

FIG. 1C shows a cross-sectional view of the handle of the catheter delivery system of FIG. 1, according to certain example embodiments;

FIGS. 2 and 2A, illustrate a magnified and side view of an implant holder of a catheter delivery system, depicting two arrangements of a pusher block and a release block around a pin wherein one arrangement (FIG. 2A) causes detachment of the implant from the implant holder, according to some example embodiments;

FIG. 2B illustrates a magnified, side view of the pusher block and a cross-sectional view along A-A plane of the pusher block of the implant holder shown in FIG. 2 of a catheter delivery system, depicting pushing surfaces and seating notch, according to certain example embodiments;

FIG. 2C illustrates a magnified and isotropic view of the release block of the implant holder shown in FIG. 2 of a catheter delivery system, depicting detaching end and legs attached to the release block, according to certain example embodiments;

FIGS. 3 and 3A, illustrate a magnified and side view of an implant holder of a catheter delivery system, depicting two arrangements of a pusher block and a release block around a pin wherein one arrangement (FIG. 3) causes detachment of the implant from the implant holder, according to certain example embodiments;

FIGS. 4 and 4A, illustrate a magnified and side view of an implant holder of a catheter delivery system, depicting two arrangements of a pusher block and a release block around a pin wherein one arrangement (FIG. 4A) causes detachment of the implant from the implant holder, according to certain example embodiments;

FIGS. 5 and 5A, illustrate a magnified and side view of an implant holder of a catheter delivery system, depicting two arrangements of a pusher block and a release block around a pin wherein one arrangement (FIG. 5 or FIG. 5A) causes detachment of the implant from the implant holder, according to certain example embodiments;

FIGS. 6 and 6A, illustrate a magnified and side view of an implant holder of a catheter delivery system, depicting two arrangements of a pusher block and a release block around a pin wherein one arrangement (FIG. 6A) causes detachment of the implant from the implant holder, according to certain example embodiments;

FIGS. 7 and 7A, illustrate a magnified and side view of an implant holder of a catheter delivery system, depicting two arrangements of a pusher block and a release block around a pin wherein one arrangement (FIG. 7A) causes detachment of the implant from the implant holder, according to certain example embodiments;

FIGS. 8 and 8A, illustrate a magnified and side view of an implant holder of a catheter delivery system, depicting two arrangements of a pusher block and a release block in the implant holder wherein one arrangement (FIG. 8A) causes detachment of the implant from the implant holder, according to certain example embodiments;

FIGS. 9 and 9A, illustrate isometric views of an implant holder of a catheter delivery system depicting a mechanism of using a movable pin according to certain example embodiments;

FIG. 9B shows an isometric view of a movable pin and FIG. 9C shows a side cross-sectional side view of a movable pin according to certain example embodiments;

FIG. 9D, illustrates a cross-sectional side view of an implant holder of a catheter delivery system depicting a mechanism of using a movable pin where the movable pin is in disappearing position, according to certain example embodiments; and

FIG. 9E, illustrates a cross-sectional side view of an implant holder of a catheter delivery system depicting a mechanism of using a movable pin where the movable pin is in visible position, according to certain example embodiments.

### DETAILED DESCRIPTION

According to the present disclosure, in some embodiments, a catheter delivery system, for trans-vascularly delivering and deploying an implant in a human or animal organ, comprises a detachment mechanism to ensure disconnect of the implant from the catheter delivery system once the implant reaches its deployment location and it is in correct position too. By definition, the detachment of the implant from the catheter delivery system is an incident after which the implant cannot be maneuvered anymore and the next process steps of retraction of the catheter from the deployment site initiate.

In one non-limiting aspect, an example catheter delivery system comprises a distal section, a middle section, and a proximal section. The proximal section remains outside the human body and comprises a handle housing that encompasses mechanisms to control the movements at the distal section of the catheter. The distal section comprises a tip, an inner shaft, a guidewire shaft, an implant holder and a capsule wherein, in a loaded state, the distal section comprises an implant too. The middle section is connected proximally with the handle housing and distally it connects to the distal section.

The capsule is a hollow, cylindrical part that is movable through movement mechanisms present in the middle section and actuated from the proximal section. The capsule provides an inside space where the implant is loaded in compressed form and the capsule helps in retaining the implant in compressed form. Usually, capsule-based catheter delivery systems are used for delivery of implants whose frame structure is made of shape memory alloys e.g., Nitinol. Such implants don't require any external force to regain their un-compressed structure. Due to shape-memory property, such implants start attaining their normal structure from an end once the capsule is moved to uncover the implant, starting from the end. The implant is situated over the guidewire shaft and in between the tip and the implant holder. The inner shaft extends longitudinally along the middle section from a proximal end of the distal section and further extends till a proximal end of the proximal section. The guidewire shaft extends from the proximal end of the proximal section till a distal end of the distal section. The guidewire shaft is connected to a threaded shaft and that connects to a rotary knob. The inner shaft is fixed to the handle housing and not movable. However, by rotating the rotary knob, the threaded shaft moves in longitudinal direction which in turn moves the guidewire shaft. In an alternative arrangement, the inner shaft is connected to the threaded shaft and that connects to the rotary knob. In this case, the guidewire shaft is fixed to the handle housing and not movable. However, by rotating the rotary knob, the threaded shaft moves in longitudinal direction which in turn moves the inner shaft.

On loading the implant on the guidewire shaft and inside the capsule, the engaging part of the frame of the implant gets engaged with the pin of the implant holder. In the implant loading procedure, the capsule is moved to compress and house the implant inside the hollow cylindrical part of the capsule. During deployment procedure, the capsule is moved to uncover the implant and the engaged part of the frame of the implant moves back to its original shape. In normal operation, this is sufficient to disengage the implant from the frame holder. However, in some cases, additional maneuvering is required to ensure detachment of the implant frame.

The implant holder is a two-part hub-like cylindrical part and situated inside the capsule at a proximal end of the distal section. First part, a pusher block, of the implant holder is attached to the inner shaft whereas a second part, a release block, is attached to the guidewire shaft. Distal side of the pusher block has a plurality of pins on its peripheral surface. The number of pins may be, for example, any number between 2-6, including 2 to 4; however other numbers of pins are also possible (e.g., greater than 6). In some examples, 1 pin may be used. These pins are, optionally, at equal distance and angle from each other circumferentially. Distal side of the release block of the implant holder is fixed to the guidewire shaft. Proximal side of the pusher block of the implant holder is fixed to the inner shaft. Proximal side of the release block has a plurality of legs where any two legs have a space between them to accommodate at least one pin located on the distal side of the pusher block. The pusher block has at least one sliding slot to accommodate at least one leg of the release block in sliding manner. In assembled state, the leg is accommodated inside the sliding slot and on moving the guidewire shaft, the release block moves in longitudinal direction and the leg travels in the sliding slot. In an alternative arrangement, in assembled state, the inner shaft is movable in longitudinal direction and the guidewire shaft is fixed. In this arrangement, on moving the inner shaft, the pusher block moves in longitudinal direction and the release block is fixed. However, the accommodation of the leg in the sliding slot remains same and the sliding movement of the leg in the sliding slot also remains the same. Usually, the pusher block has a plurality of sliding slots and the release block has a plurality of legs which are accommodated in the sliding slots in sliding manner.

In another embodiment, the release block doesn't have legs to accommodate in the sliding slots of the pusher block. However, the release block has a surface or a notch that comes in contact of the pin's peripheral surface on movement of the guidewire shaft or the inner shaft.

Further, a portion of the release block comprises at least a leg, at least a releasing part along its length and at least a detaching end. The releasing part and the detaching end, either in combination or in isolation, construct a detaching mechanism for detaching the engaging part of the frame from the pin. The length of the releasing part is sufficient to remain in contact of peripheral surface of the pin during movement of the guidewire shaft or the inner shaft. In addition, the height of the pin is either less or almost equal to the height of the detaching end. The detaching end is situated towards the distal side of the release block and connects to the releasing part and the releasing part is connected to the leg. The impact surface of the detaching end that comes in contact of the pin can be selected from a flat surface, an inclined surface, a curved surface, a concave surface, a convex surface, a V-shaped notch, a U-shaped notch, an elliptical surface, an oblong surface, an irregular geometrical surface or a combination thereof. Similarly, the edges, along the length, of the releasing part can be of different shapes selected from a linear edge or a non-linear edge selected from a tapered edge, a curved edge, a concave edge, a convex edge, an elliptical edge, an edge with at least one step change in the width of the leg in circumferential direction, an irregular geometrical edge or a combination thereof. In case of any non-linear edge of the releasing part, the direction of the non-linear edge is such that the higher extent of non-linearity is either towards the proximal section of the catheter delivery system or towards the detaching end. In another embodiment, the extent of non-linearity is equally distributed over the length of the releasing part.

In application, the pins get engaged with the frame of the implant at the time of loading the implant and they get disengaged at the time of deployment. As mentioned earlier, in some cases and due to various reasons, the implant doesn't get disengaged quickly and requires additional maneuvering to get the implant dislodged. This increases procedure time, and the positioning of the implant may also get affected. The mechanism of the two-part implant holder, as per the present disclosure, helps in ensuring the detachment of the frame of the implant from the pins of the implant holder of the catheter delivery system. On moving the guidewire shaft, the release block of the implant holder also moves in longitudinal direction and the releasing part also moves. The movement path is from one end of the releasing part to another end of the releasing part. In initial position, the pins are away from the detaching end and an engaging part of the implant frame is engaged to these pins. The engagement mechanism is simply hooking at least a part of the implant frame to the pin. On moving the release block, the detaching end moves towards the pin and due to linear or non-linear shape of the releasing part, a vertical force is applied on the engaging part of the implant frame and the engaging part comes out of the pin as the detaching end approaches the pin. In an alternative arrangement, the pusher block is movable due to longitudinally movable inner shaft and on moving the pusher block, the pin moves towards the detaching end and due to linear or non-linear shape of the releasing part, a vertical force is applied on the engaging part of the implant frame and the engaging part comes out of the pin as the detaching end approaches the pin.

In another embodiment, where the legs and/or the releasing part is not present, the release block moves on moving the guidewire shaft and the detaching end impacts the peripheral surface of the pin. Due to this sudden impact or shock, the engaging part of the frame detaches from the pin.

According to yet another embodiment of the present disclosure, the guidewire shaft is fixed and not movable in longitudinal direction. Hence, the release block attached to the guidewire shaft is also fixed. The pusher block of the implant holder is attached to the inner shaft and the pins are situated on the distal end of the pusher block. According to this embodiment, the inner shaft is movable in longitudinal direction. Hence, the pusher block is also movable. On moving the inner shaft, the pin attached to the pusher block also moves along the releasing part present on the release block and disengages the engaging part of the implant frame from the pin.

According to yet another embodiment of the present disclosure, where the detaching end doesn't play a functional role in detachment and only the releasing part is functional in the release block. Depending on the arrangement, on either moving the release block due to longitudinal movement of the guidewire shaft or on moving the pusher block due to longitudinal movement of the inner shaft, the releasing part provides a upside force that acts on the engaging part of the frame and detaches the frame from the pin.

Further, in the pusher block, optionally, a seating notch is present that provides a space around the pin for the engaging part of the frame to get accommodated. Also, due to the seating notch, a pushing surface is also created that helps in transferring a force from the catheter shaft to the implant holder and to the engaging part of the frame. The force is applied in longitudinal direction towards the distal section of the catheter delivery system by the physician and the peripheral surface of the pin provides a base support while the pushing surface is applying the force on the engaging part of the frame.

In another embodiment, the legs and the releasing parts are not present in the release block and on moving the pins situated on the pusher block, due to movement of the inner shaft, come in contact of the impact surface of the detaching end and due to this sudden impact or shock, the engaging part of the frame detaches from the pin

According to yet another embodiment of the present disclosure, the pins are movable in vertical direction through a slot in the inner shaft. According to this embodiment, the implant holder comprises three parts—a movable pin, a riser and a receiver. The riser is attached to the inner shaft and has an inclined surface facing towards the proximal section of the catheter delivery system. The receiver is also attached to the inner shaft and has another inclined surface. The another inclined surface faces towards the distal section of the catheter delivery system. The movable pin is situated in between the riser and the receiver. Shape of the movable pin is made of a cylindrical part and a hook part. The engaging part of the implant frame engages with the cylindrical part of the movable pin. For disengaging the implant from the movable pin, the movable pin is moved in vertical direction using the riser, the receiver, and longitudinal movement of the guidewire shaft. The hook part of the movable pin is parallelogram-shaped and situated at an angle with respect to the longitudinal axis of the guidewire shaft. One end of the hook part is fixed to the cylindrical part of the movable pin and an other end is situated between the riser and the receiver in such a way so that one side surface of the parallelogram is in sliding contact with the inclined surface of the riser and other side surface is in sliding contact with the inclined surface of the receiver. Also, inclined surfaces of the riser and the receiver are parallel to each other i.e., on bringing closer, the inclined surfaces of the riser and the receiver contact each other at 0° angle. In addition, the hook part of the movable pin is not fixed to any surface. On longitudinal movement of the guidewire shaft towards the distal section, the receiver moves forward and forces the hook part of the movable pin to move towards the guidewire shaft. On forward movement of the guidewire shaft, the receiver also moves forward and applies a downward force on the inclined surface of the flat part. This downward force pushes the movable pin to move downwards in the space created and the cylindrical part of the movable pin gets accommodated in the slot in the inner shaft. Similarly, on longitudinal movement of the guidewire shaft towards the proximal section, the riser also moves towards the proximal section and applies an upward force on the other inclined surface of the flat part and helps in upward movement of the movable pin through the slot in the inner shaft.

According to yet another embodiment of the present disclosure, the pin can be of various sizes and shapes, specifically selected from, but not limited to, rectangular, circular, D-shaped, oval, hexagonal, pentagonal, octagonal, triangular configurations and a combination thereof.

The materials used for fabricating such cantilever is selected from, but not limited to, stainless steel, nitinol, polyamide, polypropylene, Acrylonitrile butadiene styrene and a combination thereof.

According to yet another embodiment of the present disclosure, the implant may be, but not limited to, a stent, a valve, a mesh, a balloon, a patch, a drug-containing matrix, a shunt, or a combination thereof.

According to yet another embodiment of the present disclosure, the implant has at least one design element that can be engaged with the pin. Such design element may be, but not limited to, a hook-shaped element, a ring-shaped element, a closed ring-shaped element, an open ring-shaped element, an irregular-close-shaped element, an irregular-open-shaped element, a U-shaped element, a V-shaped element, a W-shaped element, a M-shaped element, an end part of a stent, a valve or a shunt that can be put around the pin, part of a mesh, part of a frame, or a combination thereof.

According to yet another embodiment, the implant holder comprises at least one radiopaque marker. The radiopaque marker is situated on the peripheral surface of the implant holder and its components including, but not limited to, the pusher block, the release block, the pin, the riser, the receiver, the legs, the sliding slot, the slot, the releasing part or a combination thereof.

According to yet another embodiment, shape of the radiopaque marker present on the percutaneous catheter is selected from a circle, rectangular, square, oval, hexagonal, oblong, star-shaped, diamond-shaped, a circumferential ring, an irregular-shaped circumferential ring, an incomplete circumferential ring, an incomplete irregular circumferential ring or a combination thereof.

According to yet another embodiment, the implant is used in treating any abnormality or in any medical procedure related to heart, kidney, lever, brain, pancreas, lungs, digestive system, endovascular system, any tract, duct or any conduit in animal or human body. More specifically, the implant can be deployed in an artery, vein, heart valves, esophageal duct, bile duct, urinary tract, alimentary tract, tracheobronchial tree, cerebral aqueduct or genitourinary system of an animal or human body.

In addition, the present subject matter also envisages a method for fabricating the implant holder as explained above. For the manufacturing of the implant holder, the method includes loading of a medically clean and approved workpiece in a designing instrument. According to one example of the present subject matter, the workpiece can be in shape of a hollow circular tube, or a solid cylinder, or a sheet. In some embodiments, the workpiece is prepared from a composition in powder form or prepared from a composition in liquid form. Then the required design of the implant holder is set-up or uploaded in the designing instrument, such as a computer-numerical controlled (CNC) machine for manufacturing. Subsequently, the required design is carved out of the workpiece to fabricate the implant holder. In one example, the fabrication technique used in the designing instrument is selected from laser fabrication, chemical-etching, mechanical machining, chemical machining, metal injection molding, vacuum casting, milling, photochemical-etching, electro-discharge machining, 3D-printing technique, additive manufacturing technique or a combination thereof. For instance, the implant holder is fabricated by slitting a metallic hollow circular tube with a laser beam, the laser beam following a predefined cutting contour to produce the design of the implant holder. Alternatively, the implant holder is be manufactured using 3D printing technique or additive manufacturing.

Once the implant holder has been manufactured, the undesired material is removed from the surface of the implant holder for finishing. The cleaned and finished implant holder can then be polished or coated with an appropriate coating. For example, it can be coated with an anti-reactive agent which prevents it from reacting with the atmosphere where either the implant is stored or deployed. Additionally, or alternatively, the implant holder can be covered with a medicinal substance or radiopaque substance, depending on the purpose, mode, and location of deployment of the implant holder.

3D printing technique can be selected from but not limited to Stereolithography (SLA), Digital light processing (DLP), Fused deposition modelling (FDM), Selective laser sintering (SLS), Selective laser melting (SLM), Electronic beam melting (EBM), Laminated object manufacturing (LOM), Polyjet technology or a combination of thereof.

By combining different materials and design variations explained above, a variety of configurations can be obtained with varying structure-property relationships.

Now, referring to the figures, wherein the elements are labelled with like numerals throughout the several Figures. Further, reference is made to the accompanying drawings which form a part hereof, and in which is shown by way of illustration. It is to be understood that other embodiments may be utilized, and structural changes may be made without departing from the scope of the invention.

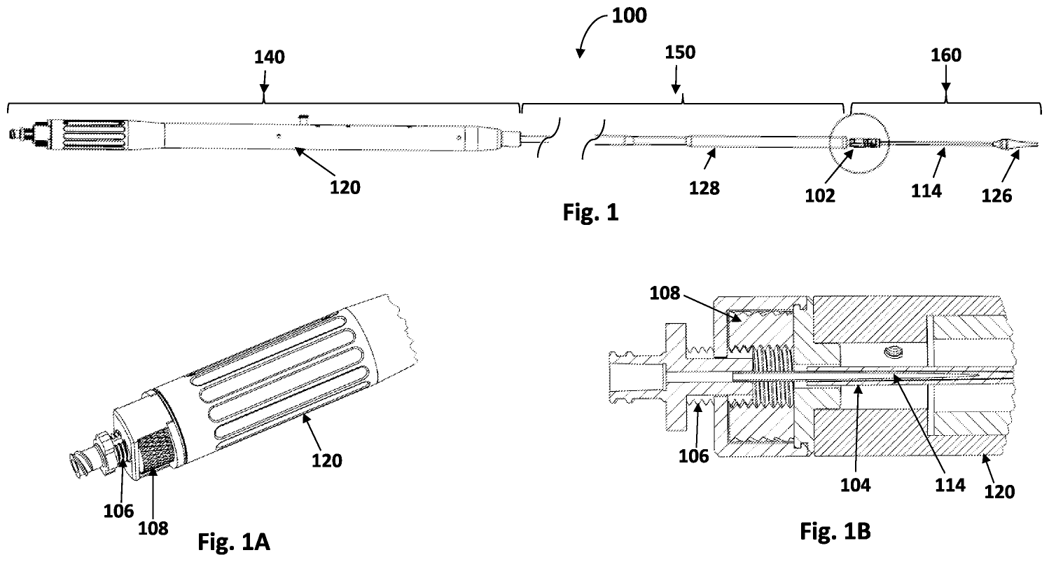

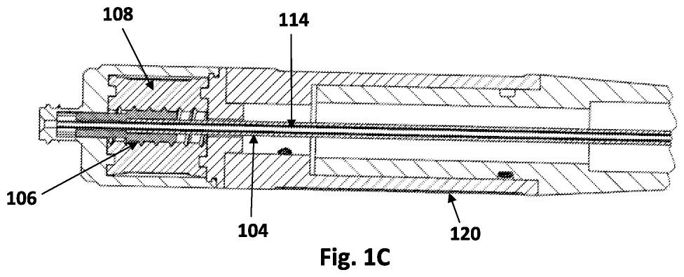

FIG. 1 represents, according to an embodiment of the present disclosure, an example catheter delivery system. The catheter delivery system (100) comprises a distal section (160), a middle section (150), and a proximal section (140). The proximal section (140) remains outside the human body and comprises a handle (120) to control the movements at the distal section (160) of the catheter. The distal section comprises a tip (126), an inner shaft (104), a guidewire shaft (114), an implant holder (102) and a capsule (128). The middle section (150) is connected proximally with the handle (120) and distally it connects to the distal section (160).

FIG. 1A represents an isometric view of a part of the handle (120) assembly that has a rotary knob (108). FIG. 1B represents a side cross-sectional view of a part of the handle (120) that comprises a rotary knob (108). The rotary knob (108) is connected to a threaded shaft (106) that is fixed to the guidewire shaft (114). The guidewire shaft (114) goes through the inner shaft (104) and extends till the tip (126) in the distal section (160). The distal section (160) mainly comprises the tip (126), the guidewire shaft (114), the implant holder (102) and the capsule (128). The capsule (128) provides an inside space where the implant is loaded in compressed form and the capsule (128) helps in retaining the implant in compressed form. The implant is situated over the guidewire shaft (114) and in between the tip (126) and the implant holder (102). The implant holder (102) is situated on the guidewire shaft (114) and situated inside the capsule (128) at a proximal end of the distal section (160). In this arrangement, the guidewire shaft (114) is movable in longitudinal direction due to rotational movement of the rotary knob (108). FIG. 1C represents a side cross-sectional view of a part of the handle (120) that comprises a rotary knob (108). The rotary knob (108) is connected to a threaded shaft (106) that is fixed to the inner shaft (104). The guidewire shaft (114) goes through the inner shaft (104) and extends till the tip (126) in the distal section (160). In this arrangement, the inner shaft (104) is movable in longitudinal direction due to rotational movement of the rotary knob (108).

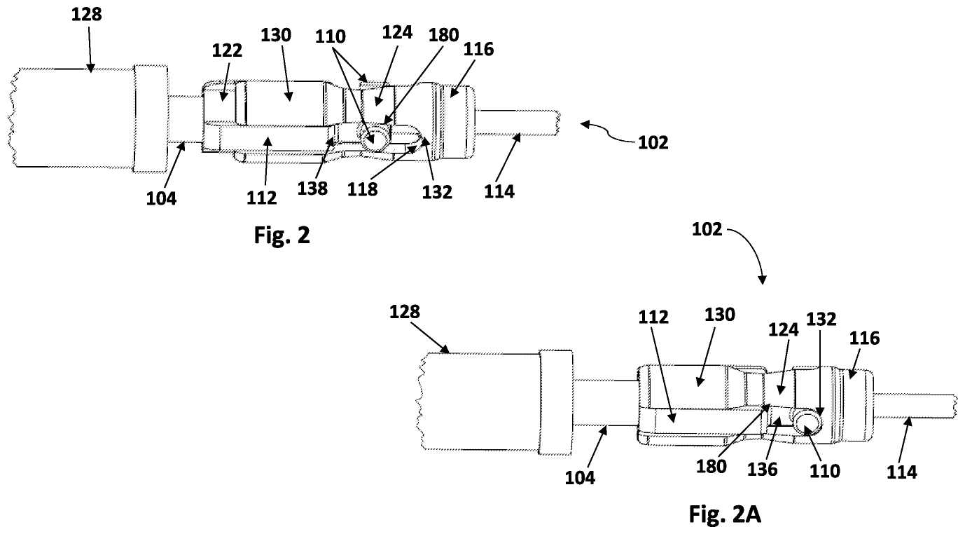

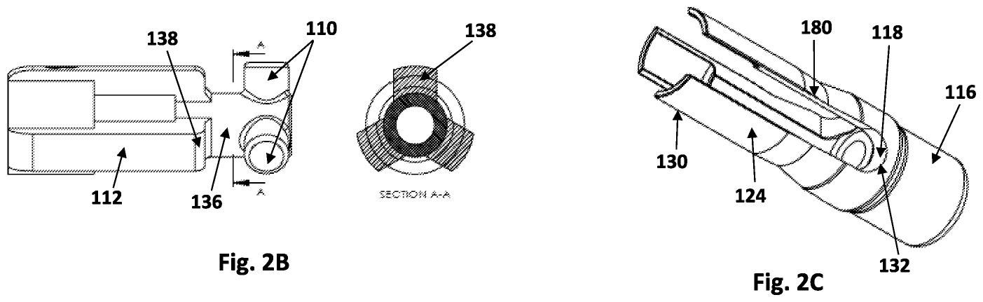

Referring to FIG. 2 and FIG. 2A, FIG. 2 shows an engaged position and FIG. 10 2A shows a position to facilitate detachment of the implant. According to an embodiment of the present disclosure, the implant holder (102) is a two-part hub-like cylindrical structure and comprises a pusher block (112) that is attached to the inner shaft (104) whereas a release block (116) is attached to the guidewire shaft (114). The release block (116) has a plurality of legs (130). The pusher block (112) has a plurality of pins (110), e.g., 3 pins that are spaced at 120 degrees circumferentially around the block, and a plurality of sliding slots (122). The legs (130) are arranged to move or otherwise engage into the sliding slots (122)—e.g., in a sliding manner. Each pin (110) is placed between two adjacent legs (130). In addition, the pusher block (112) also has a seating notch (136) around the pin (110) that accommodates the engaging part of the frame of the implant. Also, the pusher block (112) includes a pushing surface (138) that helps in transferring of a force on the engaging part of the frame wherein the force is applied by the physician in longitudinal direction of the catheter delivery system.

In an assembled state, on moving the guidewire shaft (114), the release block (116) moves in longitudinal direction and the legs (130) slide in the sliding slots (122). Further, at least one leg (130) has a releasing part (124) along its length, which may be formed or otherwise have a tapered edge (180) whose tapering height increases towards the distal end of the catheter delivery system (100). The releasing part (124) has a detaching end (132) that has a height almost equal to the height of the pin. In application, the pins (110) are engaged with frame of the implant (e.g., within 5% or less in certain example embodiments). On moving the guidewire shaft (114) in longitudinal direction, the release block (116) of the implant holder (102) also moves and so does the releasing part (124). In initial position, which is shown in FIG. 2, the pins (110) are away from the detaching end (132). On moving the release block (116), which is shown in FIG. 2A, the detaching end (132) moves towards the pin (110) and due to shape of the releasing part (124), a vertical force is applied on the engaging part of the implant frame and the engaging part comes out of the pin (110) as the detaching end (132) approaches the pin (110).

According to the embodiment depicted in FIGS. 2 and 2A, the detaching end (132) is located towards the distal section (160) of the catheter delivery system.

As shown in FIG. 2B, the pusher block (112) has a pushing surface (138) and seating notch (136) between the pin (110) and the pushing surface (138). FIG. 2C shows an impact surface (118) present at a detaching end (132) of the release block (116) that has legs (130) attached thereto. Further, at least one leg (130) has a releasing part (124) along its length, which has a tapered edge (180)

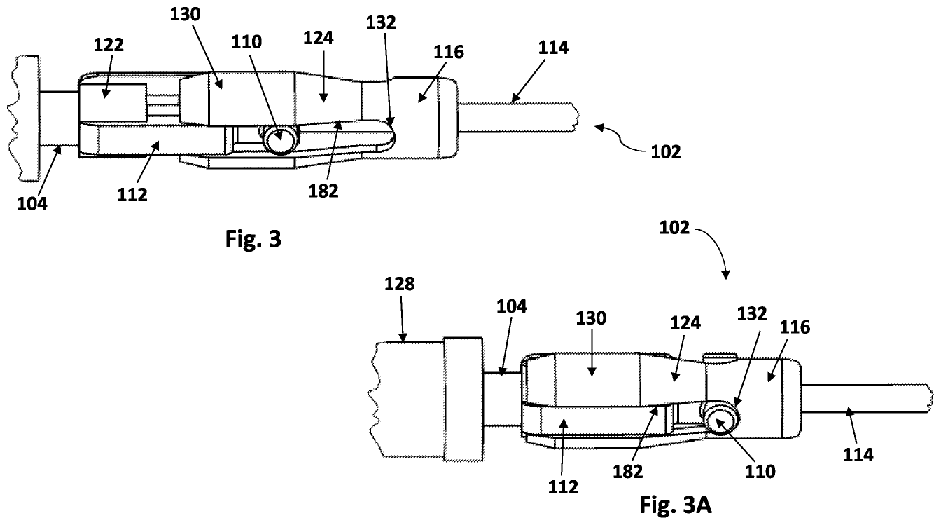

FIGS. 3 and 3A represent an embodiment where in initial position, the detaching end (132), and the pin (110) are in proximity and the detachment takes place when the pin (110) is moved away from the detaching end (132) and towards the proximal section of the catheter delivery system. Further, at least one leg (130) has a releasing part (124) along its length, which may be formed or otherwise have a reverse tapered edge (182) whose tapering height decreases towards the distal end of the catheter delivery system (100).

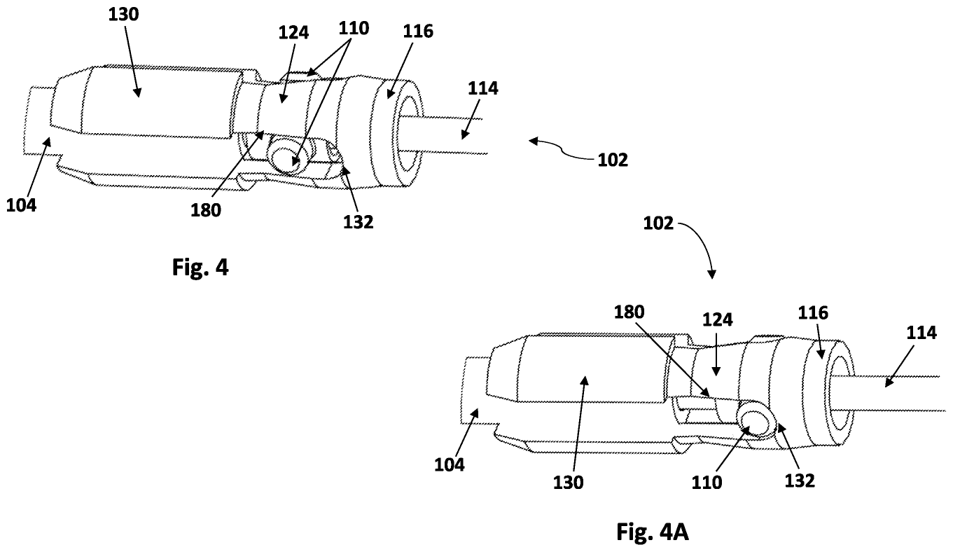

Referring to FIG. 4 and FIG. 4A, according to yet another embodiment of the present disclosure, the guidewire shaft (114) is fixed and not movable in longitudinal direction. Hence, the release block (116) attached to the guidewire shaft (114) is also fixed. According to this embodiment, the rotary knob in the handle (120) is connected to a threaded shaft that is fixed to the inner shaft (104). The pusher block (112) of the implant holder (102) is also attached to the inner shaft (104). Hence, on rotating the rotary knob, the inner shaft (104) is movable in longitudinal direction and the pusher block (112) also moves. The pins (110) attached to the pusher block (112) also moves along the releasing part (124) present on the release block (116), towards the detaching end (132), and disengages the engaging part of the implant frame from the pin (110). On moving the pusher block (112), the pins (110) move towards the detaching end (132). At least one leg (130) has the releasing part (124) along its length, which may be formed or otherwise have a tapered edge (180) whose tapering height decreases towards the distal end of the catheter delivery system (100). Due to the tapered edge (180) of the releasing part (124), a vertical force is applied on the engaging part of the implant frame and the engaging part comes out of the pin (110).

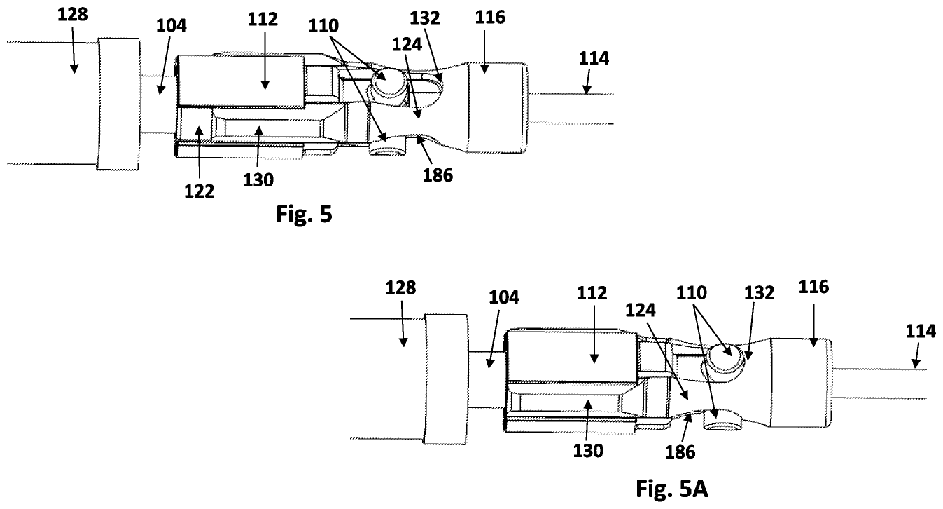

Referring to FIGS. 5 and 5A, according to another embodiment of the present disclosure, the inner shaft (104) is not movable, hence, the pins (110) attached to the pusher block (112) are also not movable. The guidewire shaft (114) is movable in longitudinal direction due to rotational motion of the rotary knob (108). The release block (116) also moves in longitudinal direction due to movement of the guidewire shaft (114) movement. The releasing part (124) along its length, which may be formed or otherwise has a concave-shaped edge (186) that applies a vertical force on the engaging part of the frame to aid its detachment from the pins (110).

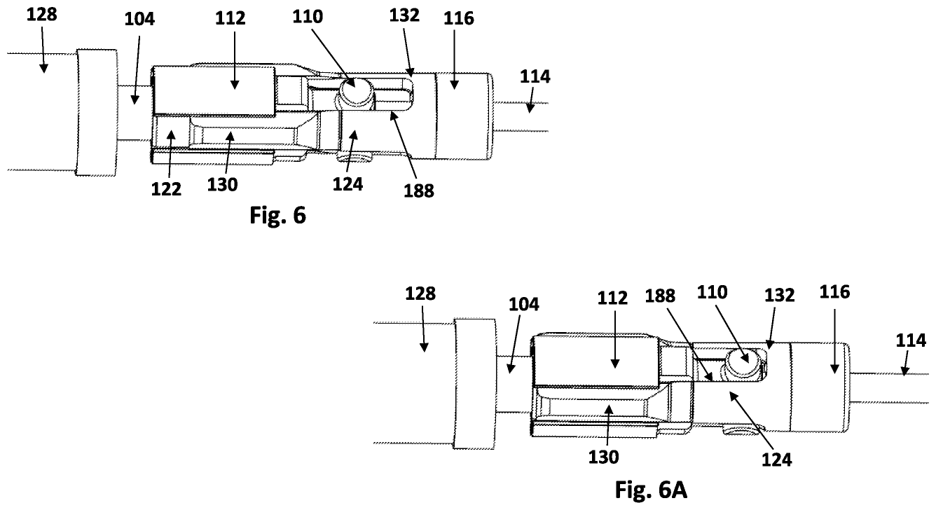

FIGS. 6 and 6A, depict yet another embodiment of the present disclosure, wherein the inner shaft (104) is not movable, hence, the pins (110) attached to the pusher block (112) are also not movable. The guidewire shaft (114) is movable in longitudinal direction due to rotational motion of the rotary knob (108). The release block (116) also moves in longitudinal direction due to movement of the guidewire shaft (114) movement. The releasing part (124) along its length, which may be formed or otherwise has a linear edge (188), with no angle change with respect to the longitudinal axis of the guidewire shaft, of the releasing part (124) applies a frictional force on the engaging part of the frame to aid its detachment from the pins (110).

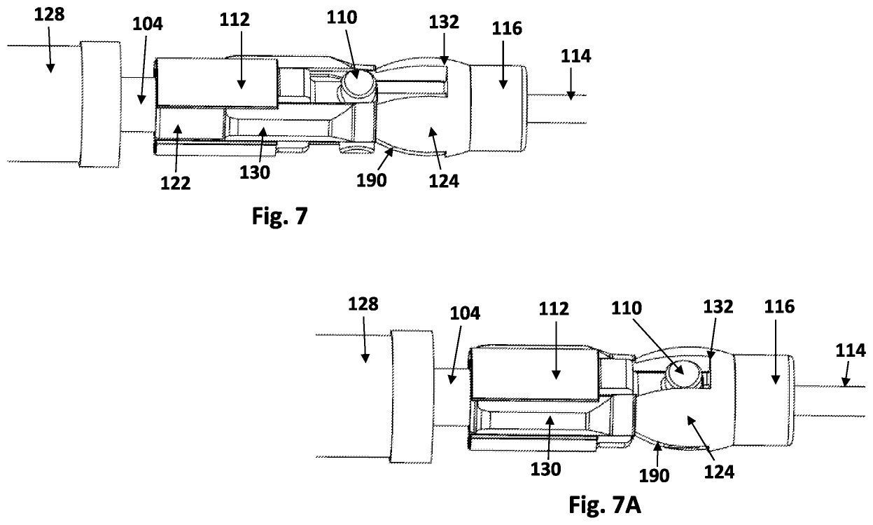

Referring to FIGS. 7 and 7A, according to yet another embodiment of the present disclosure, the inner shaft (104) is not movable, hence, the pins (110) attached to the pusher block (112) are also not movable. The guidewire shaft (114) is movable in longitudinal direction due to rotational motion of the rotary knob (108). The release block (116) also moves in longitudinal direction due to movement of the guidewire shaft (114) movement. The releasing part (124) along its length, which may be formed or otherwise has a convex-shaped edge (190) of the releasing part (124) applies an uplifting force on the engaging part of the frame to aid its detachment from the pins (110).

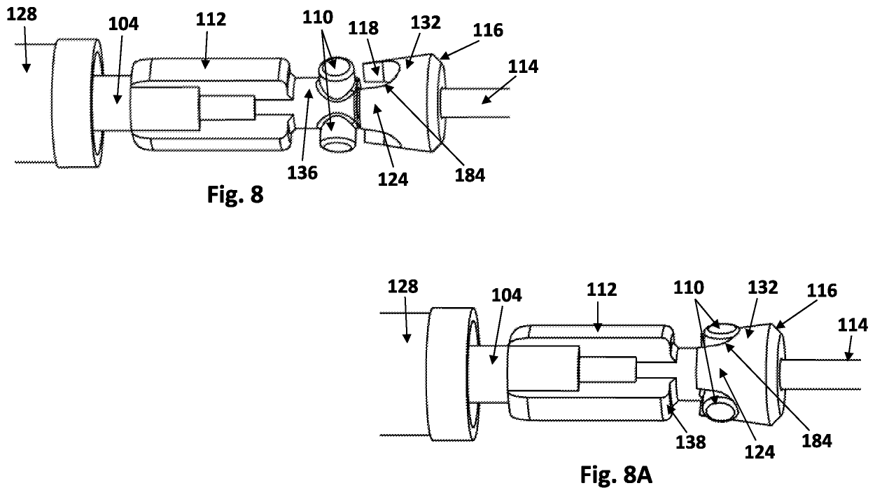

Referring to FIGS. 8 and 8A, according to yet another embodiment of the present disclosure, the inner shaft (104) is not movable, hence, the pins (110) attached to the pusher block (112) are also not movable. The guidewire shaft (114) is movable in longitudinal direction due to rotational motion of the rotary knob (108). As shown in FIGS. 8 and 8A, the release block (116), optionally, does not have the legs and/or the releasing part (e.g., as discussed in connection with other embodiments). The release block (116) is configured to move in longitudinal direction due to movement of the guidewire shaft (114) movement, and the U-shaped notch (184) of the releasing part (124) comes in contact of the peripheral surface of the pins (110) and the impact surface (118) of the detaching end (132) applies a shock or impact on the pin that aids in detachment of the engaging part of the frame the pins (110).

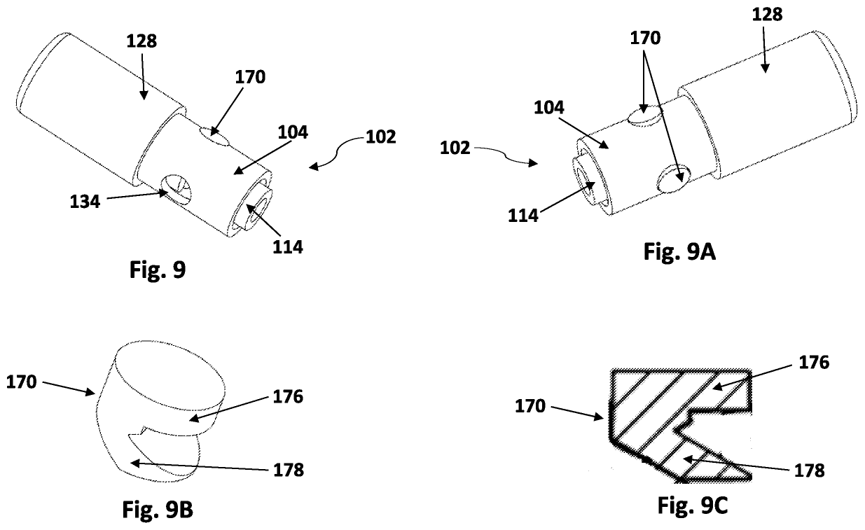

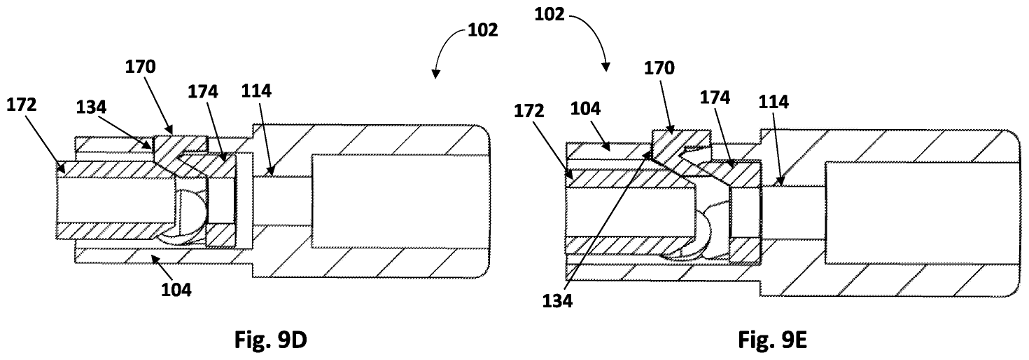

FIGS. 9 to 9E depict yet another embodiment, wherein FIG. 9 shows an isometric view of the implant holder (102) where the moveable pin (170) is below, or approximately flush with, the circumferential surface of the inner shaft (104). The moveable pin (170) is situated in a slot (134). FIG. 9 shows the slot (134) without the pin (170). And FIG. 9A shows an isometric view of the implant holder (102) where the moveable pin (170) is above the circumferential surface of the inner shaft (104). FIG. 9B shows an isometric view of the moveable pin (170) which is formed of a cylindrical part (176) and a hook part (178). FIG. 9C shows a cross-sectional view of the moveable pin (170).

FIG. 9D shows cross sectional view of the implant holder (102) in a state where the moveable pin (170) is below, or approximately flush with, the circumferential surface of the inner shaft (104). FIG. 9E shows cross sectional view of the implant holder (102) in a state where the moveable pin (170) is above the circumferential surface of the inner shaft (104).

In the embodiment shown in FIGS. 9-9E, due to longitudinal movement of the guidewire shaft (114), the movable pin (170) moves perpendicularly (e.g., transverse) to the longitudinal axis through the slot (134) in the inner shaft (104). The movable pin (170) is slidably disposed between a riser (172) and a receiver (174). The hook part (178) is parallelogram-shaped and has parallel inclined side surfaces. These side surfaces are in sliding contact with the riser (172) and the receiver (174). The riser (172) and the receiver (174) also have at least one inclined side surface which are also parallel to each other, parallel to the inclined side surfaces of the hook part (178). The riser (172) and the receiver (174) are in sliding contact with the inclined side surfaces of the hook part (178). The riser (172) and the receiver (174) both are attached to, or form part of, the guidewire shaft (114). In contrast, the movable pin (170) is separable from any part of the catheter delivery system (e.g., the movable pin is not fixed thereto and can be, as discussed herein, removed from the catheter delivery system). The cylindrical part (176) is placed inside the slot (134) in the inner shaft (104). The cylindrical part and the hook part (178) together form the movable pin (170). The movable pin (170) is situated between the riser (172) and the receiver (174). The engaging part of the implant frame is engaged with the cylindrical part (176) of the movable pin (170).

On longitudinal movement of the guidewire shaft (114) towards the distal section (160), the receiver (174) moves forward and forces the hook part (178) of the movable pin (170) to move towards the guidewire shaft (114). On forward movement of the guidewire shaft (114), the receiver (174) also moves forward and applies a downward force on the inclined surface of the hook part (178). This downward force pushes the movable pin (170) to move downwards in the space created and the cylindrical part (176) of the movable pin (170) gets accommodated in the slot (134) in the inner shaft (104). Similarly, on longitudinal movement of the guidewire shaft (114) towards the proximal section (140), the riser (172) also moves towards the proximal section (140) and applies an upward force on the other inclined surface of the hook part (178) and helps in upward movement of the movable pin (170) through the slot (134) in the inner shaft (104).

For disengaging the engaged part of the implant frame, the movable pin (170) is moved in a perpendicularly downward direction using the riser (172), the receiver (174), and longitudinal movement of the guidewire shaft (114). More specifically, when the pin (170) is up (e.g., as shown in FIG. 9A and FIG. 9 E) an implant may then be engaged to the pin (170). When the guidewire shaft (114) is moved forward, the inclined surface of the receiver (174) applies comes into contact with and applies a force on the inclined surface of the hook part (178). This causes a downward force to be applied to the pin to force it downwards. The riser (172) also correspondingly moves forward to thereby make space for the hook part (178) and allow the pin (170), and cylindrical part (176), to be pushed downwards as result. Thus, once the pin (170), and/or cylindrical part (176), is moved down into slot (134), or otherwise disappears, there will not be any pivot for an implant to remain engaged and accordingly the implant will be disengaged.

In the present description, for purpose of explanation, specific details are set forth in order to provide an understanding of the present disclosure. It will be apparent, however, to one skilled in the art that other embodiments may be practiced apart from the specific details described herein. One skilled in the art will recognize that specific details described herein may be incorporated into other embodiments.

Structures and devices shown in the figures are illustrative according to the exemplary embodiments. In other instances, detailed descriptions of well-known methods, devices, techniques, etc. are omitted so as not to obscure the description with unnecessary detail.

### LIST OF REFERENCE NUMERALS

| Reference Number | Element |
|---|---|
| 100 | Catheter |
| 102 | Implant holder |
| 104 | Inner shaft |
| 106 | Threaded shaft |
| 108 | Rotary Knob |
| 110 | Pin |
| 112 | Pusher block |
| 114 | Guidewire shaft |
| 116 | Release block |
| 118 | Impact surface |
| 120 | Handle |
| 122 | Sliding slot |
| 124 | Releasing part |
| 126 | Tip |
| 128 | Capsule |
| 130 | Leg |
| 132 | Detaching end |
| 134 | Slot |
| 136 | Seating notch |
| 138 | Pushing surface |
| 140 | Proximal section |
| 150 | Middle section |
| 160 | Distal section |
| 170 | Movable Pin |
| 172 | Riser |
| 174 | Receiver |
| 178 | Hook part |
| 176 | Cylindrical part |
| 180 | Tapered edge |
| 182 | Reverse tapered edge |
| 186 | Concave-shaped edge |
| 188 | Linear edge |
| 190 | Convex-shaped edge |
| 184 | U-shaped notch |

1. An implant detaching mechanism configured to detach an implant from an implant holder, said implant detaching mechanism comprising:
a catheter having a guidewire shaft, an inner shaft, a handle, and a rotary knob;
a release block and a pusher block, combinedly forming the implant holder;
the release block having a detaching end that has an impact surface; and
the pusher block having at least a pin configured to engage with the implant,
wherein the pusher block is fixed on the inner shaft and the release block is fixed on the guidewire shaft, and relative movement between the inner shaft and the guidewire shaft is configured to cause a corresponding movement of the release block and the pusher block;
wherein, on the pusher block and the release block being moved towards or apart from each other, a force applied on the pin or on the implant that is engaged with the pin, facilitates detachment of the implant from the pin.

2. The implant detaching mechanism as claimed in claim 1, wherein the release block has at least a leg whose one end is connected to the detaching end.

3. The implant detaching mechanism as claimed in claim 1, wherein the pusher block has at least a sliding slot.

4. The implant detaching mechanism as claimed in claim 2, wherein the leg is accommodated in the sliding slot in sliding manner.

5. The implant detaching mechanism of claim 1, wherein the leg has a releasing part along its length and of a symmetric or an asymmetric shape.

6. The implant detaching mechanism of claim 1, wherein, on the pusher block and the release block being moved towards each other, at one point, the impact surface at the detaching end of the release block impacts the pin and the resultant impact force contributes in detachment of the implant that is engaged to the pin.

7. The implant detaching mechanism of claim 1, wherein, the releasing part of the leg contacts with the implant that is engaged to the pin on moving the pusher block and the release block towards or apart from each other thereby applying a force on the engaged implant that causes detachment of the implant from the pin.

8. The implant detaching mechanism as claimed in claim 1, wherein the inner shaft in the catheter is movable in longitudinal direction on rotation of the rotary knob present in the handle of the catheter.

9. The implant detaching mechanism as claimed in claim 1, wherein the guidewire shaft in the catheter is movable in longitudinal direction on rotation of the rotary knob present in the handle of the catheter.

10. The implant detaching mechanism as claimed in claim 1, wherein the implant holder has a plurality of legs attached to the release block and a plurality of sliding slots present in the pusher block.

11. The implant detaching mechanism as claimed in claim 1, wherein the implant holder has a plurality of pins attached to the pusher block at equal distance in circumferential direction.

12. The implant detaching mechanism as claimed in claim 1, wherein the plurality of pins are attached to the pusher block at unequal distance in circumferential direction.

13. The implant detaching mechanism as claimed in claim 1, wherein the impact surface at the detaching end has a surface configuration selected from flat, curved, inclined, concave, convex, elliptical, oblong, V-shaped notch, U-shaped notch, C-shaped notch, irregular surface or a combination thereof.

14. The implant detaching mechanism as claimed in claim 1, wherein the releasing part of the leg has a linear edge or a non-linear edge selected from a tapered edge, a curved edge, a concave edge, a convex edge, an elliptical edge, an edge with at least one step change in the width of the leg in circumferential direction, an irregular shape edge or a combination thereof.

15. The implant detaching mechanism as claimed in claim 1, wherein shape of the pin present on the pusher block is selected from rectangular, circular, D-shaped, oval, hexagonal, pentagonal, octagonal, triangular configuration and a combination thereof.

16. The implant detaching mechanism as claimed in claim 1, wherein the pusher block has a pushing surface and a seating notch between the pushing surface and the pin to provide space for engaging the implant.

17. The implant detaching mechanism of claim 1, wherein the implant detaching mechanism is made of a biocompatible material selected from a group of polymers, metals, alloys, non-metals, biodegradable materials, bioresorbable materials or a combination of thereof.

18. The implant detaching mechanism of claim 1, wherein the implant holder is made of a biocompatible material selected from stainless steel, nitinol, cobalt-chromium, polyamide, polypropylene, Acrylonitrile butadiene styrene or a combination thereof.

19. The implant detaching mechanism of claim 1, wherein the implant holder has at least one radiopaque marker on its circumferential surface.

20. The implant detaching mechanism of claim 1, wherein the implant is selected from a stent, a valve, a mesh, a balloon, a patch, a drug-containing matrix, a shunt, a vena cava filter, a vascular graft, a stent graft or a combination thereof.

21. An implant detaching mechanism that is configured to detach an implant from an implant holder, comprising:
a catheter having a guidewire shaft, an inner shaft, a rotary knob, and a handle;
a riser, a receiver, and a movable pin, combinedly forming the implant holder;
the riser and the receiver including at least one inclined end, wherein the riser and the receiver are fixed on the guidewire shaft and their inclined ends face each other;
the movable pin includes a cylindrical part and a hook, wherein the cylindrical part is slidingly movable in a slot of the inner shaft; and
the hook includes two inclined surface and the hook is situated between the riser and the receiver;
wherein each inclined surface of the hook is configured to accommodate with the inclined surfaces of the riser and the receiver;
wherein, depending on the direction of the longitudinal movement of the guidewire shaft, due to rotational movement of the rotary knob in the handle, the cylindrical part of the movable pin is configured to move through the slot of the inner shaft in a vertical direction and the inclined surfaces of the hook are configured to slide on the inclined surfaces of the riser and the receiver.

22. The implant detaching mechanism of claim 21, wherein the implant detaching mechanism is made of a biocompatible material selected from a group of polymers, metals, alloys, non-metals, biodegradable materials, bioresorbable materials or a combination of thereof.

23. The implant detaching mechanism of claim 21, wherein the implant holder is made of a biocompatible material selected from stainless steel, nitinol, cobalt-chromium, polyamide, polypropylene, Acrylonitrile butadiene styrene or a combination thereof.

24. The implant detaching mechanism of claim 21, wherein the implant holder has at least one radiopaque marker on its circumferential surface.

25. The implant detaching mechanism of claim 21, wherein the implant is selected from a stent, a valve, a mesh, a balloon, a patch, a drug-containing matrix, a shunt, a vena cava filter, a vascular graft, a stent graft or a combination thereof.

26. A method of manufacturing the implant holder of claim 1, the method comprising the step of:
setting-up a design of the pusher block and the release block to be fabricated in a designing instrument;
carving the design on a work piece to fabricate the pusher block and the release block;
finishing the pusher block and the release block by removing material from a surface of the pusher block and the release block and polishing the pusher block and the release block;
arranging the pusher block on the inner shaft and the release block on the guidewire shaft to accommodating manner; and
fixing the pusher block on the inner shaft and the release block on the guidewire shaft.

27. The method as claimed in claim 26, wherein the work piece is one of a hollow circular tube, or a solid cylinder, or a sheet, or prepared from a composition in powder form or prepared from a composition in liquid form.

28. The method as claimed in claim 26, wherein the step of carving is selected from at least one of laser fabrication, chemical-etching, mechanical machining, chemical machining, metal injection molding, vacuum casting, milling, photochemical-etching, electro-discharge machining, 3D-printing technique, additive manufacturing technique or a combination thereof.
